(19) United States
(12) Patent Application Publication
(10) Pub. No.: US 2014/0323511 A1
(43) Pub. Date: Oct. 30, 2014

(54) CHEMOTHERAPEUTIC FOR INDUCING AN MSH2 DEPENDENT APOPTOTIC PATHWAY

(71) Applicant: Wake Forest University Health Sciences, Winston-Salem, NC (US)

(72) Inventors: Freddie R. Salsbury, JR., Winston-Salem, NC (US); Karin D. Scarpinato, Statesboro, GA (US); S. Bruce King, Walnut Cove, NC (US)

(73) Assignee: Wake Forest University Health Sciences, Winston-Salem, NC (US)

(21) Appl. No.: 14/193,371

(22) Filed: Feb. 28, 2014

Related U.S. Application Data
(60) Provisional application No. 61/771,335, filed on Mar. 1, 2013.

(51) Int. Cl. C07D 455/03; A61K 45/06; A61K 31/475

(52) U.S. Cl. CPC C07D 455/03; A61K 31/475; A61K 45/06
USPC 514/280; 546/54

(57) ABSTRACT

Active compounds include compounds of Formula I and Formula II are described:

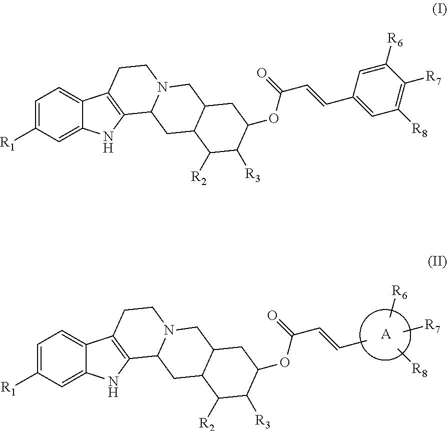

along with compositions containing the same and methods of use thereof.

### RELATED APPLICATIONS

This application claims the benefit of U.S. Provisional Patent Application Ser. No. 61/771,335, filed Mar. 1, 2013, the disclosure of which is incorporated by reference herein in its entirety.

### STATEMENT OF GOVERNMENT SUPPORT

This invention was made with Government support under Grant No. RO1 CA101829 from the National Institutes of Health. The US Government has certain rights to this invention.

### FIELD OF THE INVENTION

The present invention concerns methods and compositions for the treatment of cancer.

### BACKGROUND OF THE INVENTION

The p53 gene is one of the most studied and well-known genes. p53 plays a key role in cellular stress response mechanisms by converting a variety of different stimuli, for example, DNA damage, deregulation of transcription or replication, and oncogene transformation, into cell growth arrest or apoptosis. However, loss of p53 activity in tumors is associated with faster tumor progression and resistance to cancer treatment. See, e.g., U.S. Pat. No. 6,593,353; see also U.S. Pat. Nos. 6,998,117; 6,017,524; 5,908,750; and 5,744,310.; M. Greenblatt et al., Cancer Res. 54, 4855-4878 (1994). Hence, there is a need for new ways to treat cancers in which p53 deficiency is found.

### SUMMARY OF THE INVENTION

There is an unmet need for treating cancers in which p53 deficiency is found. One such p53 independent mechanism has recently been identified in mismatch repair proteins. Historically, mismatch repair proteins have been identified in recognizing and processing replication errors in the DNA. In addition to the recognition of mismatches, bacterial MutS protein and its eukaryotic homolog also recognize certain DNA damage. In response to such DNA damage, mismatch repair proteins activate cell death rather than damage repair. This cell death pathway is essentially p53 independent. Using a combination of computational modeling and cell biology, we have identified a “death conformation” of the MutS homologous proteins that trigger the cell death pathway. We then identified compounds, as well as the structural requirements for proposed compounds, that would fit into the “death conformation” and therefore initiate cell death in the absence of DNA. Since this cell death mechanism is p53-independent, the proposed compounds can be effective for the treatment of p53-deficient tumors. These compounds are described in our prior US Patent Application Publication No. 2010/0239522, published Sep. 23, 2010. Additional active compounds, neither suggested nor described in our prior application, are further provided herein.

Accordingly, a first aspect of the invention is a method of treating cancer in a subject in need thereof, comprising administering said subject an active compound as described herein in an amount effective to treat said cancer.

A second aspect of the present invention is a composition comprising an active agent as described herein in a pharmaceutically acceptable carrier. The composition optionally includes at least one additional chemotherapeutic agent.

A further aspect of the present invention is the use of an active compound as described above for the preparation of a medicament for the treatment of cancer as described herein.

The present invention is explained in greater detail in the drawings herein and the specification set forth below. The disclosures of all United States patent references cited herein are to be incorporated herein by reference in their entirety.

### BRIEF DESCRIPTION OF THE DRAWINGS

FIG. 1: Cell viability data for ovarian cancer cell line A2780. Compound 88 (Table 2) is represented by diamonds; compound 84 (Table 2) is represented by squares; and compound 81 (Table 2) is represented by triangles.

FIG. 2: Cell viability data for prostate cancer cell line LNCaP (with MSH2 deficiency). Compound 88 is represented by diamonds; compound 84 is represented by squares; compound 81 is represented by triangles.

FIG. 3: Cell viability data for prostate cancer cell line PC3. Compound 88 is represented by diamonds; compound 84 is represented by squares; compound 81 is represented by triangles.

FIG. 4: Cell viability data for compound 81 for endometrial cancer cells with (H; dashed line) and without (H2; solid line) MSH2 deficiency.

FIG. 5: Cell viability data for compound 84 for endometrial cancer cells with (H; dashed line) and without (H2; solid line) MSH2 deficiency.

FIG. 6: Cell viability data for compound 88 for endometrial cancer cells with (H; dashed line) and without (H2; solid line) MSH2 deficiency.

### DETAILED DESCRIPTION OF EMBODIMENTS OF THE INVENTION

“Cancer” as used herein is particularly concerned with p53 deficient tumors or cancers; or those in which p53 deficiency is found. Such cancers are known and described in, for example, U.S. Pat. Nos. 6,593,353; 6,998,117; 6,017,524; 5,908,750; and 5,744,310; and in M. Greenblatt et al., Cancer Res, 54, 4855-4878 (1994). It will be appreciated that, in some cases, cancers or tumors of a particular type can be treated by the method of the present invention even though those particular tumors are not p53 deficient.

“Treat” as used herein refers to any type of treatment that imparts a benefit to a patient afflicted with a disease, including improvement in the condition of the patient (e.g., in one or more symptoms), delay in the progression of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

“Prodrug” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound or active agent, for example, by hydrolysis in blood.

“Heterocyclic group” or “heterocyclo” as used herein alone or as part of another group, refers to an aliphatic (e.g., fully or partially saturated heterocyclo) or aromatic (e.g., heteroaryl) monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Loweralkyl” as used herein alone or as part of another group, refers to a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, and the like. The term “loweralkyl” is intended to include both substituted and unsubstituted alkyl or loweralkyl unless otherwise indicated and these groups may be substituted with from 1 to 4 groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Loweralkenyl” as used herein alone or as part of another group, refers to a straight or branched chain hydrocarbon containing from 1 to 4 carbon atoms which include 1 to 2 double bonds in the normal chain. Representative examples of alkenyl include, but are not limited to, vinyl, 2-propenyl, 3-butenyl, 2-butenyl, and the like. The term loweralkenyl” is intended to include both substituted and unsubstituted alkenyl or loweralkenyl unless otherwise indicated and these groups may be substituted with groups as described in connection with loweralkyl above.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and loweralkyl above, particularly aryl such as phenyl substituted from 1 to 3 times with loweralkoxy.

“Arylalkyl” and “arylalkynyl” as used herein alone or as part of another group, refers to an aryl group, as defined herein, appended to the parent molecular moiety through an alkyl or alkenyl group, as defined herein. Representative examples of arylalkyl include, but are not limited to, benzyl, 2-phenylethyl, 3-phenylpropyl, 2-naphth-2-ylethyl, and the like.

“Alkoxy” as used herein alone or as part of another group, refers to an alkyl or loweralkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Acyl” as used herein alone or as part of another group refers to a —C(O)R radical, where R is any suitable substituent such as aryl, alkyl, alkenyl, alkynyl, cycloalkyl or other suitable substituent as described herein.

“Halo” as used herein refers to any suitable halogen, including —F, —Cl, —Br, and —I.

“Amino” as used herein means the radical —NH₂.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the groups hydrogen, alkyl, haloalkyl, alkenyl, alkynyl, cycloalkyl, cycloalkylalkyl, aryl, arylalkyl, heterocyclo, heterocycloalkyl. —NHC(O)CH₃ is in some embodiments preferred.

“Amide” as used herein alone or as part of another group refers to a —C(O)NRₐR_{b} radical, where Rₐ and R_{b} are any suitable substituent such as alkyl, cycloalkyl, alkenyl, alkynyl or aryl.

“Sulfoxyl” as used herein refers to a compound of the formula —S(O)R, where R is any suitable substituent such as alkyl, cycloalkyl, alkenyl, alkynyl or aryl.

“Sulfonyl as used herein refers to a compound of the formula —S(O)(O)R, where R is any suitable substituent such as alkyl, cycloalkyl, alkenyl, alkynyl or aryl.

“Sulfonate” as used herein refers to a compounnd of the formula —S(O)(O)OR, where R is any suitable substituent such as alkyl, cycloalkyl, alkenyl, alkynyl or aryl.

“Sulfonic acid as used herein refers to a compound of the formula —S(O)(O)OH.

“Sulfonamide” as used herein alone or as part of another group refers to a —S(O)₂NRₐR_{b} radical, where Rₐ and R_{b} are any suitable substituent such as H, alkyl, cycloalkyl, alkenyl, alkynyl or aryl.

The present invention is primarily concerned with the treatment of human subjects (including both male and female subjects; and including infant, juvenile, adolescent, adult, and geriatric subjects), but the invention may also be carried out on animal subjects, particularly mammalian subjects such as mice, rats, dogs, cats, livestock and horses for veterinary purposes, and for drug screening and drug development purposes.

### 1. Active Compounds.

Active compounds include compounds of Formula I:

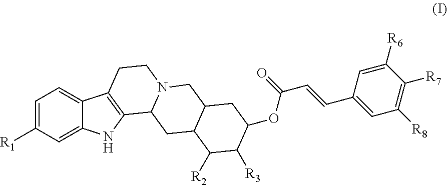

wherein:

R₁ is H, loweralkyl, or loweralkoxy (preferably H or methoxy, most preferably methoxy);

R₂ is —C(O)OR″ or —NHC(O)OR″, where R″ is H or loweralkyl (preferably H or methyl);

R₃ is hydroxyl or loweralkoxy (preferably hydroxyl or methoxy);

R₆, R₇, and R₈ are each independently selected from the group consisting of H, OH, OCH₃, COON, —OC(O)R₉, amino, alkylamino, arylalkylamino, disubstituted amino, acylamino, amide, sulfoxyl, sulfonyl, sulfonate, sulfonic acid, and sulfonamide,

at least one of R₆, R₇ and R₈ is selected from the group consisting of —OC(O)R₉, amino, alkylamino, arylalkylamino, disubstituted amino, acylamino, amide, sulfoxyl, sulfonyl, sulfonate, sulfonic acid, and sulfonamide (preferably —OC(O)R₉, amino, alkylamino, arylalkylamino, disubstituted amino, acylamino, or amide);

R₉ is loweralkyl,

or a pharmaceutically acceptable salt thereof.

Examples of the foregoing include, but are not limited to:

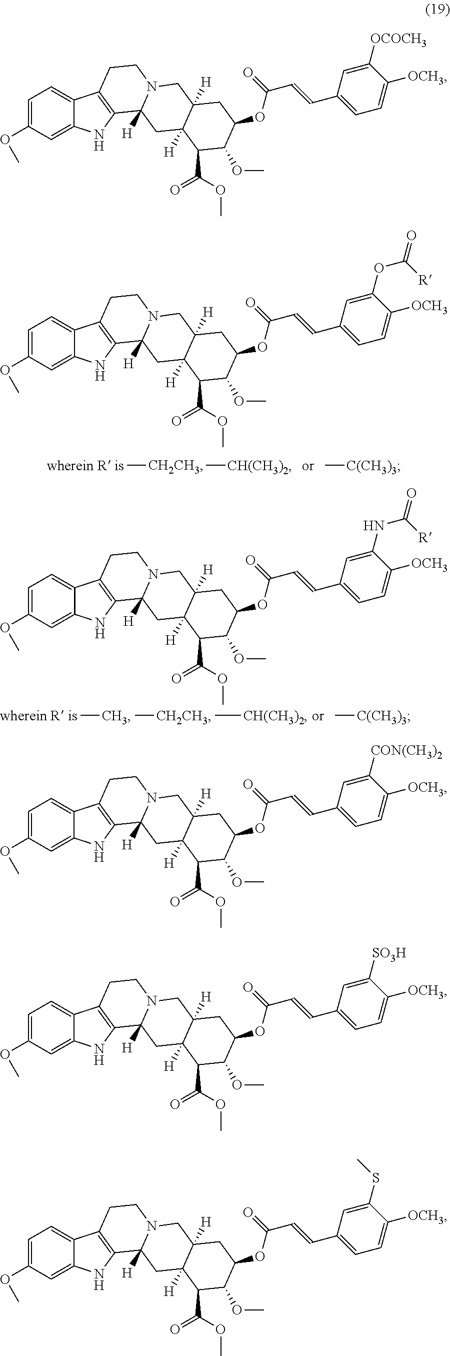

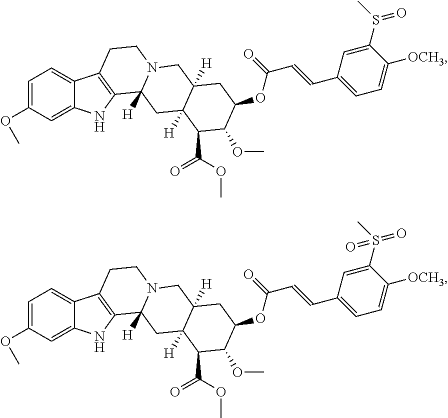 [SMILES: COC(=O)[C@H]1[C@H]2C[C@@H]3c4[nH]c5cc(OC)ccc5c4CCN3C[C@H]2C[C@@H](OC(=O)/C=C/c2ccc(OC)c(S(C)=O)c2)[C@@H]1OC ; COC(=O)[C@H]1[C@H]2C[C@@H]3c4[nH]c5cc(OC)ccc5c4CCN3C[C@H]2C[C@@H](OC(=O)/C=C/c2ccc(OC)c(S(C)(=O)=O)c2)[C@@H]1OC]

and pharmaceutically acceptable salts thereof.

Active compounds also include compounds of Formula II:

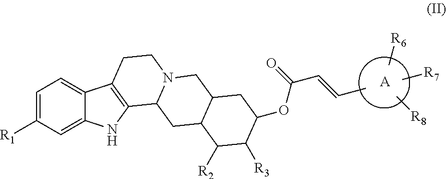

wherein:

ring A is heterocyclic group;

R₁ is H, loweralkyl, or loweralkoxy (preferably H or methoxy, most preferably methoxy);

R₂ is —C(O)OR″ or —NHC(O)OR″, where R″ is H or loweralkyl (preferably H or methyl);

R₃ is hydroxyl or loweralkoxy (preferably hydroxyl or methoxy);

R₆, R₇, and R₈ are each (when present) independently selected from the group consisting of H, OH, OCH₃, COON, —OC(O)R₉, amino, alkylamino, arylalkylamino, disubstituted amino, acylamino, amide, sulfoxyl, sulfonyl, sulfonate, sulfonic acid, and sulfonamide,

R₉ is loweralkyl,

or a pharmaceutically acceptable salt thereof.

In some embodiments of compounds of Formula II, ring A is selected from the group consisting of azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxazole, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, and trithiane.

In some embodiments of compounds of Formul II, least one of R₆, R₇ and R₈ is selected from the group consisting of —OC(O)R₉, amino, alkylamino, arylalkylamino, disubstituted amino, acylamino, amide, sulfoxyl, sulfonyl, sulfonate, sulfonic acid, and sulfonamide (preferably —OC(O)R₉, amino, alkylamino, arylalkylamino, disubstituted amino, acylamino, or amide).

Examples of compounds of Formula II include, but are not limited to, the following:

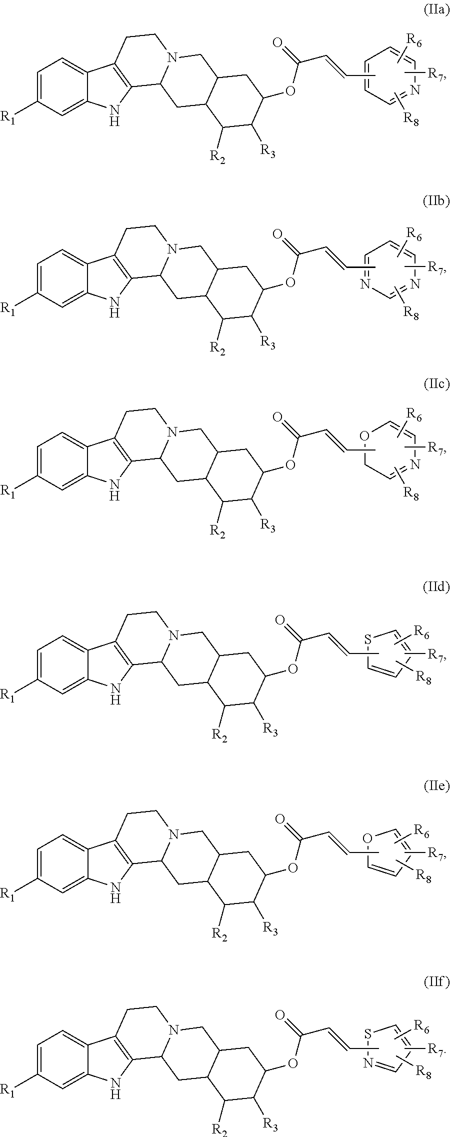

Where R₁, R₂, R₃, R₆, R₇, and R₈ (when present) are as given above.

Particular examples of the foregoing include, but are not limited to, the following:

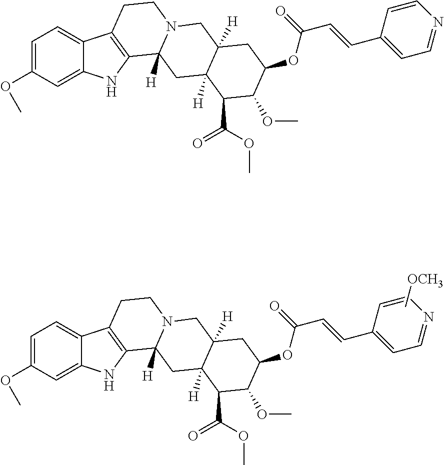

and pharmaceutically acceptable salts thereof.

Active compounds of the present invention also include a compound of the formula:

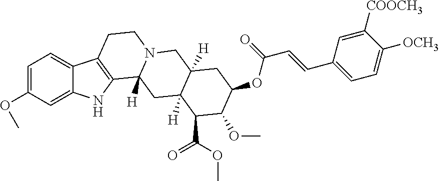 [SMILES: COC(=O)c1cc(/C=C/C(=O)O[C@@H]2C[C@@H]3CN4CCc5c([nH]c6cc(OC)ccc56)[C@H]4C[C@@H]3[C@H](C(=O)OC)[C@H]2OC)ccc1OC]

or a pharmaceutically acceptable salt thereof.

Active compounds can be made according to known techniques, the methods described herein, or variations thereof that will be apparent to those skilled in the art. For example, Oxidation of methyl reserpate will provide the corresponding ketone. Condensation of this ketone with various synthetic and commercially available hydrazines will generate the hydrazones.

The active compounds disclosed herein can, as noted above, be prepared in the form of pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

### 2. Pharmaceutical Formulations.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound as described herein, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compounds, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

### 3. Dosage and Routes of Administration.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 or 1 to about 20 or 50 mg/kg may be used, with all weights being calculated based upon the weight of the active compound, including the cases where a salt is employed. The frequency and duration of the treatment can be once or twice per day for a period of two to four months or more, or until the condition is essentially controlled.

Active compounds can be administered as prodrugs of the foregoing. A thorough discussion of prodrugs is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

### 4. Combination Treatments.

In another embodiment, it is envisioned to use an active compound of the invention in combination with other therapeutic modalities, in like manner as described in U.S. Pat. No. 6,566,395 to Moran. Thus, the active agents described herein may be administered in administered in combination with one or more additional chemotherapeutic agents; and/or in combination with radiation therapy; and/or in combination with ablative or partially ablative surgery. Examples of additional chemotherapeutic agents include but are not limited to the group consisting of androgens, asparaginase, azathioprine, 5-azacitidine, BCG, bleomycin, busulfan, carbetimer, carboplatin chlorambucil, cisplatin, corticosteroids, cyclophosphamide, cytarabine, dacarbazine, dactinomycin, daunomycin, doxorubicin, epirubicin, estrogens, etoposide, fadrazole, 5-fluorouracil, gemcitabine, hydroxyurea, ifosfamide, interferon alpha, interferon beta, interferon gamma, an interleukin, isotretinoin, lomustine, melphalan, 6-mercaptopurine, methotrexate, mitomycin-c, mitotane, mitoxantrone, paclitaxel, pentostatin, procabazine, progestins, rituximab, streptozocin, tamoxifen, taxotere, teniposide, thioguanine, thiotepa, topotecan, toremifene, tretinoin, uracil mustard, vinblastine, vincristine and vinorelbine.

The present invention is explained in greater detail in the following non-limiting Examples.

### EXAMPLES

Mismatch repair proteins function in a number of cellular processes, of which cytotoxic response and apoptosis is one of them. The precise mechanism of their involvement in apoptosis remains to be determined and appears to be dependent on the type of damage to the cell (REF). We previously used computational modeling to identify a distinct, death-inducing conformation of the mismatch repair proteins MSH2/MSH6 (Salsbury et al., 2006) that can be targeted for the treatment of cancer cells (Vasilyeva et al., 2009, 2010). The MSH2/MSH6-dependent cell death pathway activates pro-apoptotic proteins, such as caspase-3 and caspase-9, but is independent of many proteins commonly mutated in cancer, e.g. p53 (Topping et al., 2009).

Our initial computational approach that examined hundreds of possible compounds targeting the MSH2/MSH6 death conformation identified two Rauwolfia alkaloids, reserpine and rescinnamine that are expected to mimic the response induced by cisplatin (Vasilyeva et al., 2009). Rescinnamine showed the best cytotoxic activity by activating pro-apoptotic proteins (Vasilyeva et al., 2010). It is a member of an understudied group of compounds previously used as anti-hypertensive drugs in the 1950s and 60s that target the angiotensin-converting enzyme (ACE). Rescinnamine has lower neurotoxicity, but also lower antihypertensive efficacy than its predecessor, reserpine (Fife et al., 1960). The drug has no known genotoxic or chromosome destabilizing effects (Brambilla & Martinelli, 2006). Neither one of these compounds had been systematically studied as anticancer agents.

We here demonstrate that the anti-hypertensive effects limit the usefulness of rescinnamine as a tumor-inhibiting drug, and employ computational simulation and organic synthesis to modify the compound toward the development of a more efficient anti-cancer drug. Application of the palladium-mediated Heck coupling reaction to this structurally complicated alkaloid natural product allows for the efficient preparation of numerous rescinnamne derivatives to test computational predictions and build a structure-activity relationship (SAR).

Computationally, we continued our successful protocol of using structures obtained from molecular dynamics simulations along with docking into structures representative of the death-inducing conformations (Vasilyeva et al., 2009). However, we improved upon our previous methods in three ways. First, we used structures obtained via cluster analysis of a longer-time scale simulation of hMSH2/6 (Negureanu and Salsbury, 2012). Second, we upgraded to Autodock4 (Morris et al, 2009), with its improved force field, from Autodock3 (Morris et al, 1998). Third, we focused on modification of reserpine and rescinnamine, rather than docking of new compounds. This latter required development of a library of modified compounds, which were then docked and ranked.

### Materials and Methods

Cell Biology.

HEC59 cells (msh2 deficient) and the paired cell line HEC59 chr. that restores the MSH2-deficiency via chromosome transfer have been extensively characterized (Umar et al., 1997). Cells were grown in standard growth media (DMEM-F12+10% FBS). Cells were plated in microtiter plates at an appropriate concentration in 100 μl media and incubated overnight. Media was replaced with media containing drug and allowed to incubate for 24 hours at indicated concentrations. Untreated cells received fresh media with vehicle only. One solution reagent (CellTiter 96)(r) Aqueous One Solution) was added to existing media (20 μl/well) and allowed to incubate 3-4 hrs. A plate reader was used to record the absorbance at 490 nm. Assays were performed at least in triplicates. Cell viability at each concentration was analyzed for IC₅₀ values using GraphPad Prism 4™. Graphs represent mean values and standard deviations.

Xenograft.

SW416 or HEC59 cells in PBS mixed with Matrigel (1:1; BD Biosciences) were subcutaneously injected into the flank of nude donor mice. Tumors were grown for up to 3 weeks. Mice were euthanized, tumors excised, minced into 3 mm pieces, and surgically implanted into the right flank of acceptor mice (10 per group). Isoflurane anesthesia was provided during tumor inoculation. Injection of compounds was started 3 days following tumor implantation to allow recovery from surgery. Compounds were given intraperitoneally, in a volume of 0.5 ml/mouse with a ¼ inch, 23-gauge needle in a 6 ml plastic syringe. Mice were monitored based on survival and body weight. Tumor size, measured by caliper, and body weight were monitored daily for 57 days, and the prolongation of median survival time after ip treatment determined. Any animals showing signs of distress, unnatural movements, severe loss of appetite, severe signs of hypotension, tumor size of 1000 g, or weight loss exceeding 10% before the end of the study were euthanized. Tumors were measured twice a week for each group. Tumor volume is calculated as length(mm)×width (mm)². Initial measurements were performed when the tumor reached 150-200 mg. Tumor weight (in mg) is calculated as tumor weight (mg)−(length(mm) of tumor×width (mm) of tumor²)/2.

Chemistry-General.

Reagents were obtained from commercial sources and used without additional purification. Extraction and flash chromatography solvents were technical grade. Flash chromatography was carried out using a Biotage SP1-B2A0/HPFC System. Analytical thin layer chromatography (TLC) was performed as silica gel plates with C-4 Spectroline 254 indicator. Visualization was accomplished with UV light and 20% phosphomolybdic acid solution in EtOH. LC-MS, ESI-MS and HPLC solvents were HPLC grade. Melting points were determined on a MeI-Temp apparatus. ¹H NMR and ¹³CNMR spectra were taken in commercial deuterated solvents and recorded on a Bruker Advance 300 MHz and Bruker DRX-500 spectrometer using a 5 mm TBI probe equipped with z axis gradients. Probe temperature was regulated at 25° C. All data was collected and processed with Topspin 1.3 using standard Bruker processing parameters. Chemical shifts (6) are given in ppm; multiplicities are indicated by s (singlet), d (doublet), t (triplet), q (quartet), m (multiplet), dd (doublet of doublet) and br (broad).

Acryloyl Reserpate (1) (SA-I-03).

Dry distilled pyridine (791 mg, 0.808 mL, 10 mmole) was added to a mixture of methyl reserpate (300 mg, 0.74 mmole) and acryloyl chloride (135.77 mg, 0.123 mL, 1.5 mmole) was added and stirred under nitrogen at room temperature for 74 hr. Excess pyridine was evaporated and the residue was taken up in chloroform (50 mL) and the organic layer washed with water (3×20 mL) and brine, the organic layer was dried with anhydrous Na₂SO₄, filtered and evaporated under reduced pressure to give a residue that was purified by flash chromatography (silica gel, chloroform/methanol, 3%, R_{f} 0.3) giving a solid rinsed with methanol to afford 1 (120 mg, 35%) as white crystals: mp>260° C.; ¹H-NMR (300 MHz, CDCl₃, δ): 7.80 (s, 1H), 7.31 (d, 1H, J=8.52 Hz), 6.81 (s, 1H, J=1.95 Hz), 6.76 (d, 1H, J=8.53 Hz), 6.45 (d, 1H, J=17.32 Hz), 6.15 (dd, 1H, J=17.31& 17.30 Hz), 5.87 (d, 1H, J=10.4 Hz), 4.90-4.82 (m, 1H), 4.42 (s, 1H), 3.82 (s, 3H), 3.80 (s, 3H), 3.48 (s, 3H), 3.19-3.14 (m, 2H), 3.02-2.98 (m, 1H), 2.69-2.60 (m, 2H), 2.49-2.39 (m, 2H), 2.26-2.17 (m, 2H), 2.04-1.78 (m, 5H); ¹³C-NMR (75 MHz, CDCl₃, δ): 172.77, 165.42, 156.10, 136.31, 130.79, 130.45, 128.63, 122.13, 118.45. 108.92, 107.92, 95.18, 77.69, 77.54, 60.68, 55.76, 53.68, 51.75, 51.72, 51.17, 48.96, 33.93, 32.25, 29.47, 24.17, 16.75; ESI-MS: m/z=469.2 (M⁺+H); Anal. Calcd. for C₂₆H₃₂N₂O₆.0.3H₂O (474.487) C, 65.81; H, 6.94; N, 5.90. Found: C, 65.60; H, 6.83; N, 6.01.

General Procedure for the Synthesis of Rescinnamine Derivatives (2-7)

A mixture of the aryl iodide (0.507 mmole), acryloyl reserpate (300 mg, 0.641 mmole), Et₃N (0.06 g, 0.09 mL, 0.645 mmol) and Pd(OAc)₂ (1.23 mg, 0.005 mmole) in the presence of tri-o-tolylphosphine (4.5 mg, 0.021 mmole) in acetonitrile (20 mL) was heated with stirring in a capped sealed glass tube under argon at 90° C. for 48 hrs. After cooling, the solvent was removed under reduced pressure; the residue was dissolved in chloroform (30 mL) and washed with water (3×15 mL) and brine. The organic layer was dried with anhydrous sodium sulfate, filtered and evaporated under reduced pressure to give a residue that was purified by flash chromatography to afford a solid that was recrystallized as described below:

For 2: (R₁═COOCH₃, R₂═OCH₃, SA-I-012):

White crystals recrystallized from methanol, yield=180 mg (44%), (chloroform/methanol 3%, R_{f}=0.7), mp=215-217° C.; ¹H NMR (500 MHz, CDCl₃, δ): 8.03 (s, 1H), 7.69 (s, 1H), 7.67-7.66 (m, 2H), 7.35 (d, 1H, J=8.52), 7.02 (d, 1H, J=8.77 Hz), 6.86 (s, 1H), 6.79 (d, 1H, J=8.5 Hz), 6.40 (d, 1H, J=15.98), 4.97-4.92 (m, 1H), 4.47 (s, 1H), 3.97 (s, 3H), 3.95 (s, 3H), 3.86 (s, 3H), 3.84 (s, 3H), 3.54 (s, 3H), 3.19-3.18 (m, 2H), 3.06-3.03 (m, 1H), 2.69-2.66 (m, 1H), 2.52-2.45 (m, 3H), 2.30-2.25 (m, 2H), 1.98-1.80 (m, 5H); ¹³C NMR (125 MHz, CDCl₃, δ): 172.94, 166.41, 166.11, 160.58, 156.03, 143.66, 136.30, 133.49, 131.63, 130.43, 126.48, 122.05, 120.28, 118.55, 116.84, 112.34, 108.94, 107.85, 95.04, 65.93, 60.97, 56.26, 55.76, 53.69, 52.40, 51.76, 51.17, 48.92, 33.92, 32.25, 31.08, 29.62, 24.21, 16.75, 15.33; ESI-MS: m/z=633.4 (M⁺+H); Anal. Calcd for C₃₅H₄₀N₂O₉ (623.70): C, 66.44; H, 6.37; N, 4.43. Found: C, 66.16; H, 6.46; N, 4.41.

For 3 (R₁═OCH₃, R₂═COOH, SA-I-013):

Yellow crystals recrystallized from diethyl ether, yield=150 mg (38%), (chloroform/methanol 8%, R_{f}=0.1), mp=228-230° C.; ¹H NMR (300 MHz, DMSO, δ): 10.62 (s, 1H), 7.70 (d, 1H, J=15.97 Hz), 7.58 (d, 1H, J=7.83 Hz), 7.47 (s, 1H), 7.32 (d, 1H, J=7.83 Hz), 7.24 (d, 1H, J=8.55 Hz), 6.82 (s, 1H), 6.79 (d, 1H, J=15.91 Hz), 6.63 (d, 1H, J=8.57 Hz), 4.90-4.82 (m, 1H), 4.53 (s, 1H), 3.88 (s, 3H), 3.79 (s, 3H), 3.75 (s, 3H), 3.43 (s, 3H), 3.17-3.11 (m, 3H), 2.97-2.84 (m, 3H), 2.72-2.66 (m, 1H), 2.24 (d, 1H), 1.87-1.73 (m, 6H); ¹³C NMR (75 MHz, DMSO, δ): 171.81, 170.22, 165.82, 157.10, 155.80, 144.75, 137.20, 136.09, 129.43, 128.36, 121.26, 120.57, 118.97, 118.67, 111.83, 108.99, 105.82, 94.88, 79.40, 77.59, 76.63, 60.55, 55.99, 55.40, 53.47, 52.29, 50.73, 50.06, 48.87, 32.21, 31.29, 28.94, 23.05, 16.00; ESI-MS: m/z=619.3 (M⁺+H); Anal. Calcd for C₃₄H₃₈N₂O₉.2.5H₂O (663.71): C, 61.53; H, 6.53; N, 4.22. Found: C, 61.41; H, 7.11; N, 4.00.

For 4 (R₁═COOH, R₂═OCH₃, SA-I-015):

Pale yellow solid recrystallized from diethyl ether, yield=108 mg (27%), (chloroform/methanol 8%, R_{f}=0.1), mp=249-250° C.; ¹H NMR (500 MHz, DMSO, δ): 10.61 (s, 1H), 8.32 (s, 1H), 7.93 (s, 1H), 7.87 (d, 1H, J=8.57 Hz), 7.70 (d, 1H, J=15.96 Hz), 7.24 (d, 1H, J=8.44 Hz), 7.16 (d, 1H, J=8.66 Hz), 6.82 (s, 1H), 6.63 (d, 1H, J=8.34 Hz), 6.55 (d, 1H, J=15.99 Hz), 4.81-4.79 (m, 1H), 4.49 (s, 1H), 3.87 (s, 3H), 3.80 (s, 3H), 3.75 (s, 3H), 3.42 (s, 3H), 3.14-3.13 (m, 3H), 2.94-2.85 (m, 3H), 2.68-2.66 (m, 1H), 2.21-2.16 (m, 1H), 1.99-1.74 (m, 6H); ¹³C NMR (125 MHz, DMSO, δ): 172.07, 168.17, 166.19, 159.75, 155.79, 144.34, 137.13, 132.76, 130.86, 126.43, 123.99, 121.87, 118.53, 116.70, 113.15, 108.81, 106.24, 95.28, 79.59, 77.91, 77.02, 60.64, 56.41, 55.67, 53.97, 52.31, 51.31, 50.96, 48.69, 33.17, 32.12, 29.67, 23.67, 16.57; ESI-MS: m/z=619.3 (M⁺+H); Anal. Calcd for C₃₄H₃₈N₂O₉.3H₂O (672.72): C, 60.70; H, 6.59; N, 4.16. Found: C, 60.76; H, 6.39; N, 4.07.

For 5 (R₁═OCH₃, R₂═OH, SA-I-016):

White flakes from chloroform, yield=108 mg (27%), (chloroform/methanol 4%, R_{f}=0.3), mp=259-260° C.; ¹H NMR (300 MHz, DMSO, δ): 10.49 (s, 1H), 9.61 (s, br, 1H), 7.62 (d, 1H, J=15.87 Hz), 7.35 (s, 1H), 7.21 (d, 1H, J=8.48 Hz), 7.14 (d, 1H, J=8.35 Hz), 6.82-6.79 (m, 2H), 6.61 (d, 1H, J=8.49 Hz), 6.50 (d, 1H, J=15.89 Hz), 4.87-4.78 (m, 1H), 4.33 (s, 1H), 3.83 (s, 3H), 3.78 (s, 3H), 3.75 (s, 3H), 3.41 (s, 3H), 3.04-3.01 (m, 2H), 2.87-2.81 (m, 2H), 2.66-2.63 (m, 1H), 2.36-2.31 (m, 2H), 2.19-2.12 (m, 1H), 2.04-2.00 (m, 1H), 1.93-1.69 (m, 5H); ¹³C NMR (75 MHz, DMSO, δ): 172.88, 166.49, 156.23, 148.72, 145.98, 144.79, 136.34, 130.55, 128.02, 122.22, 121.86, 118.55, 116.23, 113.18, 110.59, 109.05, 108.13, 95.20, 77.84, 65.84, 60.77, 55.99, 55.82, 53.71, 51.78, 51.19, 49.06, 34.04, 32.34, 29.69, 24.28, 16.81, 15.26; ESI-MS: m/z=591.2 (M⁺+H).

For 6 (R₁═OH, R₂═OCH₃, SA-I-020):

Yellow solid recrystallized from diethyl ether, yield=174 mg (43%), (chloroform/methanol 3%, R_{f}=0.3), mp=180-182° C.; ¹H NMR (300 MHz, CDCl₃, δ): 7.77 (s, 1H), 7.63 (d, 1H, J=15.92 Hz), 7.36 (d, 1H, J=8.54 Hz), 7.32 (d, 1H, J=8.54 Hz), 7.27 (s, 1H), 6.97 (d, 1H, J=8.56 Hz), 6.82 (s, 1H), 6.76 (d, 1H, J=8.53 Hz), 6.33 (d, 1H, J=15.92 Hz), 4.95-4.87 (m, 1H), 4.42 (s, 1H), 3.85 (s, 3H), 3.82 (s, 3H), 3.81 (s, 3H), 3.50 (s, 3H), 3.49-3.44 (m; 2H), 3.18-3.14 (m, 2H), 3.02-2.97 (m, 1H), 2.67-2.62 (dd, 1H), 2.49-2.39 (m, 3H), 2.33 (s, 3H), 2.04-1.78 (m, 5H); ¹³C NMR (125 MHz, CDCl₃, δ): 172.86, 168.88, 166.34, 156.18, 152.97, 143.86, 140.06, 136.38, 130.57, 127.82, 127.50, 122.21, 121.94, 118.51, 116.76, 112.35, 108.98, 107.99, 95, 25, 77.87, 65.84, 60.79, 56.02, 55.82, 53.74, 51.79, 51.23, 49.04, 34.03, 32.32, 29.69, 24.25, 20.62, 16.81, 15.27; ESI-MS: m/z=6.33.3 (M⁺+H); Anal. Calcd for C₃₅H₄₀N₂O₉.0.25H₂O (637.20): C, 65.97; H, 6.41; N, 4.40. Found: C, 65.74; H, 6.55; N, 4.49.

For 7 (R₁═OCOCH₃, R₂═OCH₃, SA-I-019):

Yellow solid recrystallized from diethyl ether, yield=174 mg (43%), (chloroform/methanol 3%, R_{f}=0.3), mp=180-182° C.; ¹H NMR (300 MHz, CDCl₃, δ): 7.77 (s, 1H), 7.63 (d, 1H, J=15.92 Hz), 7.36 (d, 1H, J=8.54 Hz), 7.32 (d, 1H, J=8.54 Hz), 7.27 (s, 1H), 6.97 (d, 1H, J=8.56 Hz), 6.82 (s, 1H), 6.76 (d, 1H, J=8.53 Hz), 6.33 (d, 1H, J=15.92 Hz), 4.95-4.87 (m, 1H), 4.42 (s, 1H), 3.85 (s, 3H), 3.82 (s, 3H), 3.81 (s, 3H), 3.50 (s, 3H), 3.49-3.44 (m, 2H), 3.18-3.14 (m, 2H), 3.02-2.97 (m, 1H), 2.67-2.62 (dd, 1H), 2.49-2.39 (m, 3H), 2.33 (s, 3H), 2.04-1.78 (m, 5H); ¹³C NMR (125 MHz, CDCl₃, 8): 172.86, 168.88, 166.34, 156.18, 152.97, 143.86, 140.06, 136.38, 130.57, 127.82, 127.50, 122.21, 121.94, 118.51, 116.76, 112.35, 108.98, 107.99, 95.25, 77.87, 65.84, 60.79, 56.02, 55.82, 53.74, 51.79, 51.23, 49.04, 34.03, 32.32, 29.69, 24.25, 20.62, 16.81, 15.27; ESI-MS: m/z=6.33.3 (M⁺+H); Anal. Calcd for C₃₅H₄₀N₂O₉.0.25H₂O (637.20): C, 65.97; H, 6.41; N, 4.40. Found: C, 65.74; H, 6.55; N, 4.49.

Propionoyl Reserpate (8) (SA-I-058):

Using the same procedure for 1 substituting propionyl chloride for acryloyl chloride yields a residue that was purified by flash chromatography (silica gel, chloroform/methanol, 3%, R_{f} 0.2) to give a solid that was rinsed with methanol/diethylether (1:1) to afford 8 (317 mg, 56%) as a fine light yellow powder: mp=258° C.; ¹H-NMR (300 MHz, CDCl₃, δ): 7.60 (br, s, 1H), 7.32 (d, 1H, J=8.54 Hz), 6.82 (s, 1H), 6.76 (d, 1H, J=8.59 Hz), 4.81-4.72 (m, 1H), 4.41 (s, 1H), 3.83 (s, 3H), 3.80 (s, 3H), 3.74-3.71 (m, 1H), 3.49 (s, 3H), 3.24-3.10 (m, 2H), 3.03-2.88 (m, 2H), 2.63-2.58 (m, 1H), 2.50-2.31 (m, 2 μl), 2.28-2.12 (m, 4H), 2.03-1.75 (m, 4H), 1.17 (t, 3H, J=1.35, 7.39 Hz); ¹³C-NMR (75 MHz, CDCl₃, δ): 173.79, 172.52, 156.21, 136.42, 130.53, 122.27, 118.52, 109.02, 108.05, 95.34, 77.74, 77.32, 60.61, 55.83, 53.73, 51.78, 51.69, 51.22, 49.05, 34.01, 32.33, 29.55, 28.03, 24.25, 16.80, 9.16; ESI-MS: m/z=471.3 (M⁺+H); Anal. Calcd, for C₂₆H₃₄N₂O₆. (470.56) C, 66.36; H, 7.28; N, 5.95. Found: C, 66.08; H, 7.44; N, 5.87.

5-iodo-2-methoxyphenyl acetate (9) (SA-I-017):

2-Methoxyphenylacetate (28.5 g, 171.87 mmol) was added to a mixture of iodine (17.46 g, 68.75 mmol) and HIO₃ (7.18 g, 40.8 mmol) in glacial acetic acid (190 mL), chloroform (50 mL), water (65 mL) and concentrated sulfuric acid (2 mL) and this mixture was stirred for 24 h at 40° C. Chloroform (50 mL) and water (30 mL) were added and the mixture was washed with dilute NaHSO₃ (3×) and water. The organic layer was dried with magnesium sulfate and the organic solvent was removed under vacuum to leave a residue that was recrystallized from ethanol to afford 9 as white crystals (11.7 g, 23%); (chloroform/methanol 2%, R_{f}, 0.3), m.p=75° C.; ¹H-NMR (300 MHz, CDCl₃, δ): 7.49-7.46 (dd, 1H J=8.63, 2.15 Hz), 7.34 (d, 1H, J=2.17 Hz), 6.73 (d, 1H, J=8.66 Hz), 3.79 (s, 3H), 2.29 (s, 3H); ¹³C-NMR (75 MHz, CDCl₃, δ): 168.58, 151.37, 140.48, 135.71, 131.62, 114.33, 81.32, 55.98, 20.55; ESI-MS: m/z=292.9 (M⁺); Anal. Calcd. for C₉H₉IO₃.0.5CH₃COOH (322.0964) C, 37.29; H, 3.44. Found: C, 37.92; H, 3.09.

N-hydroxy-5-iodo-2-methoxybenzamide (10, SA-I-028).

Separate solutions of hydroxylamine hydrochloride (605 mg, 8.60 mmole) in methyl alcohol (75 mL) and potassium hydroxide (1.2 g, 18.81 mmole) in methanol (50 mL) are prepared at the boiling point of the solvent. Both are cooled to 30-40° C. and the one containing alkali was added with shaking to the solution of hydroxylamine; any excessive rise of temperature during the addition is prevented by occasional cooling in an ice bath. After all the alkali has been added, the mixture is allowed to stand in an ice bath for 5 min to ensure complete precipitation of potassium chloride followed by filtration. The filtrate was added to methyl 5-iodo-2-methoxybenzoate (500 mg, 1.72 mmole) and the mixture was heated to reflux for 6 h and cooled to room temperature. The mixture was acidified with glacial acetic acid until the pH was about 6 and concentrated to remove the solvents. The residue was mixed with EtOAc (100 mL) and water (80 mL) was added to get a clear solution. The organic layer was separated, and the aqueous solution was extracted with EtOAc (2×50 mL). The combined organic phases were washed with brine, dried over anhydrous sodium sulfate, and concentrated to afford a white solid residue, recrystallized from chloroform to afford 9 as white crystals (362 mg, 73% yield) (Ethyl acetate/Hexane 1:5, R_{f}=0.3); mp=157-158° C.; ¹H-NMR (300 MHz, DMSO, δ): 10.68 (s, 1H), 9.16 (br, s, 1H), 7.82-7.72 (m, 2H), 7.01-6.93 (d, 1H, J=8.62 Hz), 3.81 (s, 3H); ¹³C-NMR (75 MHz, DMSO, δ): 161.50, 156.48, 139.93, 137.55, 124.89, 114.64, 82.73, 55.85; ESI-MS: m/z=294.0 (M⁺+1); Anal. Calcd. for C₈H₈INO₃ (293.0585) C, 32.79; H, 2.75; N, 4.78. Found: C, 32.82; H, 2.61; N, 4.79.

N-hydroxy-4-iodo-2-methoxybenzamide (11, SA-I-38):

This compound was prepared using the same procedure for 7 with methyl-4-iodo-2-methoxybenzoate as substrate to afford an off-white solid that was recrystallized from ethyl acetate/n-hexane to yield 10 as off-white crystals (102 mg, 21%); (Ethyl acetate/Hexane 1:5, R_{f}=0.4); mp=105-106° C.; ¹H-NMR (300 MHz, CDCl₃, δ): 10.24 (s, br, 1H), 7.82 (d, 1H, J=8.23 Hz), 7.41 (d, 1H, J=8.12 Hz), 7.28 (s, 1H), 3.95 (s, 3H); ¹³C-NMR (75 MHz, CDCl₃, δ): 163.13, 157.03, 132.89, 130.94, 120.77, 118.01, 99.66, 56.52; ESI-MS: m/z=294.0 (M⁺+1); Anal. Calcd. for C₈H₈₁NO₃.0.1CH₃COOC₂H₅ (301.8690) C, 33.42; H, 2.94; N, 4.64. Found: C, 33.48; H, 2.76; N, 4.57.

General Procedure for Synthesis of Rescinnamine Derivatives (12-15):

A similar Heck coupling procedure without a phosphine ligand using the appropriate substituted aryl iodides affords the corresponding substituted rescinnamine derivatives (12-15).

For 12 (SA-I-29):

Yellowish brown solid recrystallized from diethyl ether, yield=132 mg (35%), (chloroform/methanol 2%, R_{f}=0.2), mp=202-203° C.; ¹H NMR (300 MHz, CDCl₃, δ): 7.66 (s, 1H), 7.63 (d, J=15.91 Hz, 1H), 7.32 (d, J=8.54 Hz, 1H), 6.94-6.90 (m, 2H), 6.82 (s, 1H), 6.74 (d, J=8.70 Hz, 2H), 6.27 (d, J=15.88 Hz, 1H), 4.96-4.87 (m, 1H), 4.44 (s, 1H), 3.88 (s, 3H), 3.83 (s, 3H), 3.81 (s, 3H), 3.51 (s, 3H), 3.42-3.44 (m, 1H), 3.20-3.15 (m, 2H), 3.04-2.89 (m, 2H), 2.73-2.61 (m, 1H), 2.50-2.42 (m, 2H), 2.29-2.17 (m, 2H), 2.05-1.77 (m, 4H), 1.28-1.18 (m, 2H); ¹³C NMR (75 MHz, CDCl₃, δ): 172.92, 166.68, 156.23, 149.42, 145.33, 136.54, 136.38, 130.51, 127.47, 122.21, 120.30, 118.53, 115.41, 113.13, 110.18, 109.05, 108.06, 95.23, 77.88, 65.84, 60.75, 55.83, 55.59, 53.75, 51.78, 51.21, 49.08, 34.04, 32.32, 29.74, 24.27, 16.81, 15.26; ESI-MS: m/z=590 (M⁺+1), 295 (M⁺+2). HRMS-ESI⁺ (m/z): [M+H]⁺ calcd for C₃₃H₄₀N₃O₇, 590.2866. found, 590.2846; Anal. Calcd for C₃₃H₃₉N₃O₇.0.8H₂O (604.09): C, 65.61; H, 6.77; N, 6.96. Found: C, 65.27; H, 6.63; N, 6.87.

For 13 (SA-I-39):

Pale yellow flakes recrystallized from diethyl ether, yield=153 mg (41%), (chloroform/methanol 2%, R_{f}=0.4), mp=210-213° C.; ¹H NMR (300 MHz, CDCl₃, δ): 7.81 (s, br, 1H), 7.61 (d, J=15.8.4 Hz, 1H), 7.31 (d, J=8.54 Hz, 1H), 7.01 (d, J=7.98 Hz, 1H), 6.96 (s, 1H), 6.82 (s, 1H), 6.75 (d, J=8.54 Hz, 1H), 6.66 (d, J=7.97 Hz, 1H), 6.23 (d, J=15.83 Hz, 1H), 4.95-4.80 (m, 1H), 4.43 (s, br, 1H), 4.14 (s, 1H), 3.88 (s, 3H), 3.82 (s, 3H), 3.80 (s, 3H), 3.50 (s, 3H), 3.18-3.13 (m, 2H), 3.03-2.93 (m, 2H), 2.67-2.58 (m, 1H), 2.49-2.40 (m, 2H), 2.27-2.16 (m, 2H), 1.92-1.77 (m, 5H); ¹³C NMR (75 MHz, CDCl₃, δ): 172.99, 166.94, 156.15, 146.97, 145.77, 139.20, 136.36, 130.51, 128.67, 124.60, 123.34, 122.15, 118.52, 114.06, 113.39, 108.98, 107.96, 95.19, 60.77, 55.82, 55.50, 53.73, 51.82, 51.19, 49.03, 41.81, 34.01, 32.30, 30.95, 29.77, 24.24, 16.78, 13.85; ESI-MS: nilz=590 (M⁺+1), Anal. Calcd for C₃₃H₃₉N₃O₇.0.6H₂O (600.49): C, 66.01; H, 6.75; N, 7.00. Found: C, 66.11; H, 6.64; N, 6.71.

For 14 (SA-I-21):

Grayish white solid, yield=289 mg (70%), recrystallized from benzene, (chloroform/methanol 6%, R_{f}=0.3), mp=245-255° C.; ¹H NMR (500 MHz, DMSO, 8): 10.96 (s, 1H), 7.57 (s, 1H), 7.49 (d, 1H, J=15.78 Hz), 7.37-7.34 (m, 2H), 7.23 (s, 1H), 6.86 (s, 1H), 6.71 (d, 1H, J=8.49 Hz), 6.27 (d, 1H, J=15.79 Hz), 5.06 (s, br, 1H), 5.06 (s, 1H), 4.69-4.72 (m, 1H), 3.81 (s, 3H), 3.79 (s, 3H), 3.77 (s, 3H), 3.74-3.68 (m, 2H), 3.65-3.57 (m, 1H), 3.44 (s, 3H), 3.17-3.07 (m, 1H), 2.84-2.99 (m, 1H), 2.85-2.82 (m, 1H), 2.73-2.69 (m, 1H), 2.30-2.08 (m, 5H), 1.94-1.83 (m, 2H); ¹³C NMR (125 MHz, DMSO, 8): 171.76, 171.24, 165.79, 157.77, 155.92, 149.02, 147.59, 145.74, 137.31, 128.29, 124.66, 122.56, 120.84, 120.64, 118.63, 118.35, 112.76, 112.08, 109.04, 105.34, 94.82, 77.12, 75.66, 60.23, 55.66, 55.25, 54.32, 51.95, 50.16, 31.27, 30.34, 28.34, 22.49, 15.49; ESI-MS: m/z=635.2 (M⁺+H); HRMS: [M⁺+H—CH₃] calcd for C₃₄H₃₉N₂O₁₀, 635.2605. found, 635.2582; Anal. Calcd for C₃₅H₄₀N₂O₁₀.2.5H₂O (679.711): C, 61.71; H, 6.25; N, 4.23. Found: C, 61.84; H, 5.87; N, 4.28.

For 15 (SA-I-27):

Dark yellow solid, yield=140 mg (35%), recrystallized from diethyl ether, (chloroform/methanol 5%, R_{f}=0.1), mp=229-231° C.; ¹H NMR (300 MHz, DMSO, δ): 10.49 (s, 1H), 8.96 (s, br, 1H), 7.63 (d, 1H, J=15.81 Hz), 7.20 (d, 1H, J=8.57 Hz), 7.06 (s, 2H), 6.80 (s, 1H), 6.62-6.54 (m, 2H), 4.89-4.76 (m, 1H), 4.35 (s, 1H), 3.82 (s, 6H), 3.79 (s, 3H), 3.75 (s, 3H), 3.42 (s, 3H), 3.10-2.97 (m, 3H), 2.89-2.79 (m, 3H), 2.69-2.60 (m, 3H), 2.42-2.14 (m, 5H); ¹³C NMR (75 MHz, DMSO, 8): 171.61, 165.98, 155.05, 148.01, 145.61, 138.35, 136.35, 131.20, 128.52, 124.37, 121.65, 117.81, 114.96, 108.01, 106.32, 105.92, 94.77, 77.54, 76.40, 64.87, 60.05, 56.09, 55.18, 53.32, 51.74, 51.05, 50.74, 48.62, 33.19, 32.14, 29.53, 23.46, 16.44, 15.12; ESI-MS: m/z=621.3 (M⁺+H).

### Molecular Docking

The molecular docking was performed in four phases; structural model generation, ligand library generation, receptor grid generation and finally docking of the ligand library into the receptor grid. Models for the structures were generated using molecular dynamics as in our previous works (Vasilyeva, A et al, 2009; Vasilyeva, A et al, 2010; Salsbury. 2-1-). However, for this work more extensive simulations were used, The details of these simulations are reported elsewhere (Negureanu and Salsbury, 2012). In short, the simulations were four 20 ns NPT all-atom simulations based on the human MSH2/6 crystal structure (Morris et al, 2009), with the (1,2)G cross-link, which is the predominate damage due to cisplatin. The structure selected was the median structure of the most populated cluster found from all-atom RMSD-based clustering (Negureanu and Salsbury, 2012), The appropriate pdbqs file was generated with the DNA removed, so that just the protein remained, using defaults from Autodock4.

Libraries for docking was based on the core of the rescinnamine structure (Formula A). All possible derivatives were made based on this structure (Formula A), with R1, R2 and R3 selected from H, OH, COOH, NH2, and OCH3.

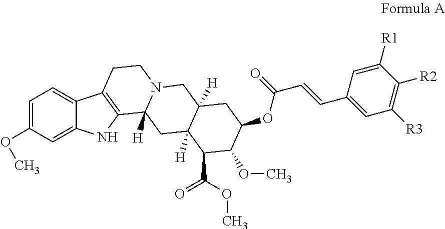

Also, a structure without the modified phenyl ring was docked (compound SA-I-03 Table I). The best of these compounds were suggested for chemical synthesis. We also explored larger libraries, and ones with other functional modifications, but these were not readily synthesized and so are not discussed herein. Autodock tools were used to generate 3D pdbq files with charges and the correct number of rotatable bonds.

The grids for docking was generated using Autodock4 with the grid centered at the position of the platinum atom in the full protein-DNA complex, however, the DNA was removed prior to grid generation. Cubic 22.5 A grids were generated for electrostatics, and vdW parameters for C, S, O, N and polar H with a grid spacing of 0.375 A.

The dockings were performed using Autodock4's defaults for it's Lamarckian Genetic Algorithm with a population size of 150, a maximum number of energy evaluations of 5 million and a maximum number of generations of 27000. Each derivative was subjected to 256 runs and ranked according to the best predicted Ki.

### Results

Rescinnamine as a First Lead in Inducing MSH2-Dependent Cell Death.

We had previously shown that reserpine and rescinnamine are capable of inducing cell death in an MSH2-dependent manner (Vasilyeva 2009, 2010). Further experiments demonstrated that this indole alkaloid is capable of overcoming resistance to cisplatin in ovarian cancer cells (data not shown). These initial results suggested rescinnamine to be a good lead toward the development of an effective cancer drug. When applied to a xenograft model, however, the hypotensive activity of the drug prevented administration of statistically effective doses, though a significant trend toward tumor inhibition is observed (data not shown). Since none of the commercially available reserpine analogs showed much promise in inducing MSH2-dependent cell death (Vasilyeva et al., 2010), we engaged computational modeling and chemical synthesis to generate novel rescinnamine analogs.

In keeping with our previous studies, we computationally-modeled derivatives of rescinnamine. These computational studies could be used to determine if the activity of rescinnamine could be improved by modification of functional groups on the ring systems, or alternatively not perturbed while perturbing the hypotensive effects of rescinnamine. In this work, the former, changes in activity, is explored.

Although there are many possible functionalization sites, and many possible R-groups, in order to avoid a combinatorial explosion computationally, and to restrict our work to experimentally feasible compounds, we restricted ourselves to derivatives that could be readily made as discussed in the methods. The best of these were suggested for chemical synthesis.

Organic Synthesis of Rescinnamine Analogs.

Promising lead molecules that were predicted to induce MSH2-dependent cell death and had a favorable partition coefficient were considered for chemical synthesis (Table 1).

TABLE 1
Rescinnamine Analogs.

| | | | 50IC | |
|---|---|---|---|---|
| Entry | Entry | | MSH2 | MSH2 |
| OLD | NEW | Compound | deficient | proficient |
| SA-I-03 | 1 | 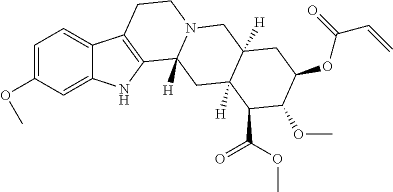 [SMILES: C=CC(=O)O[C@@H]1C[C@@H]2CN3CCc4c([nH]c5cc(OC)ccc45)[C@H]3C[C@@H]2[C@H](C(=O)OC)[C@H]1OC] | 20.7 | 20.37 |
| SA-I-058 | 8 | 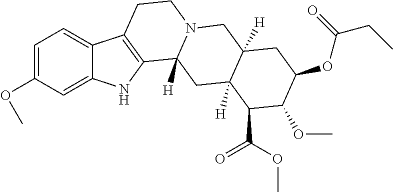 [SMILES: CCC(=O)O[C@@H]1C[C@@H]2CN3CCc4c([nH]c5cc(OC)ccc45)[C@H]3C[C@@H]2[C@H](C(=O)OC)[C@H]1OC] | NA | NA |
| SA-I-012 | 2 | 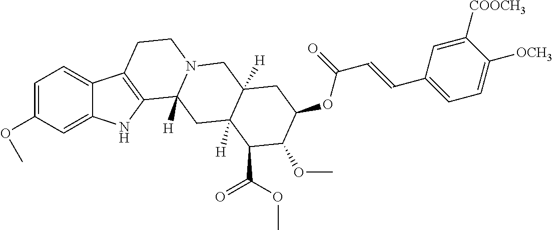 [SMILES: COC(=O)c1cc(/C=C/C(=O)O[C@@H]2C[C@@H]3CN4CCc5c([nH]c6cc(OC)ccc56)[C@H]4C[C@@H]3[C@H](C(=O)OC)[C@H]2OC)ccc1OC] | 50.72 | 48.91 |
| SA-I-013 | 3 | 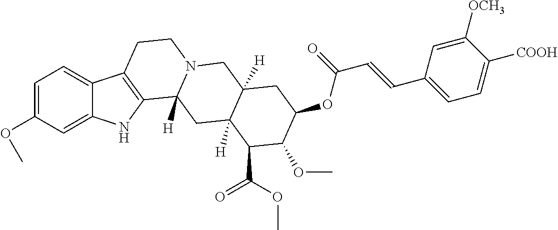 [SMILES: COC(=O)[C@H]1[C@H]2C[C@@H]3c4[nH]c5cc(OC)ccc5c4CCN3C[C@H]2C[C@@H](OC(=O)/C=C/c2ccc(C(=O)O)c(OC)c2)[C@@H]1OC] | 163.1 | 1223 |
| SA-I-015 | 4 | 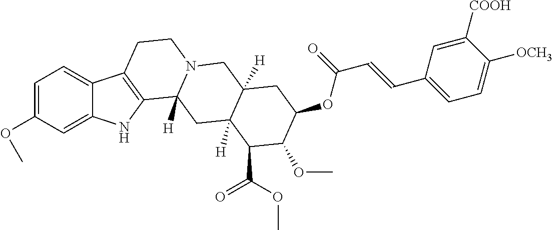 [SMILES: COC(=O)[C@H]1[C@H]2C[C@@H]3c4[nH]c5cc(OC)ccc5c4CCN3C[C@H]2C[C@@H](OC(=O)/C=C/c2ccc(OC)c(C(=O)O)c2)[C@@H]1OC] | 89.29 | 84.23 |
| SA-I-016 | 5 | 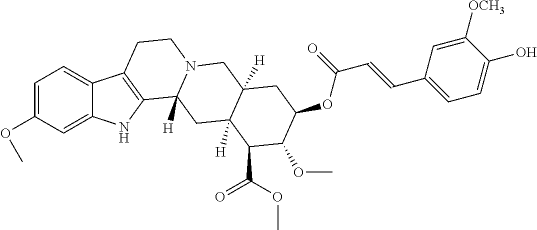 [SMILES: COC(=O)[C@H]1[C@H]2C[C@@H]3c4[nH]c5cc(OC)ccc5c4CCN3C[C@H]2C[C@@H](OC(=O)/C=C/c2ccc(O)c(OC)c2)[C@@H]1OC] | NA | NA |
| SA-I-020 | 6 | 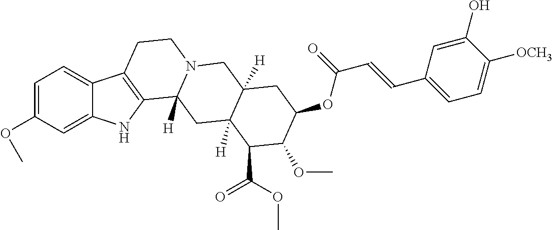 [SMILES: COC(=O)[C@H]1[C@H]2C[C@@H]3c4[nH]c5cc(OC)ccc5c4CCN3C[C@H]2C[C@@H](OC(=O)/C=C/c2ccc(OC)c(O)c2)[C@@H]1OC] | 22.32 | 21.29 |
| SA-I-019 | 7 | 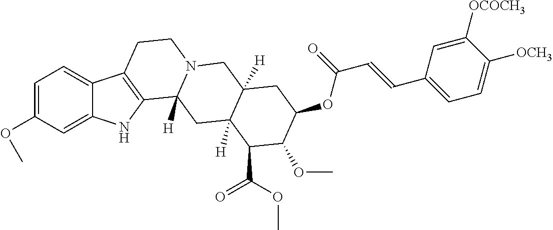 | 39.28 | NA |
| SA-I-021 | 14 | 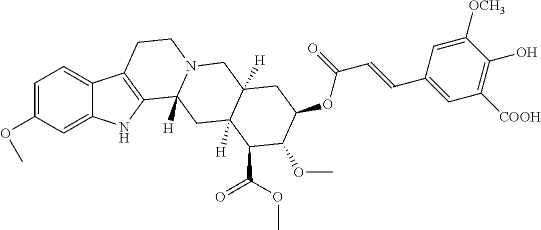 [SMILES: COC(=O)[C@H]1[C@H]2C[C@@H]3c4[nH]c5cc(OC)ccc5c4CCN3C[C@H]2C[C@@H](OC(=O)/C=C/c2cc(OC)c(O)c(C(=O)O)c2)[C@@H]1OC] | NA | NA |
| SA-I-027 | 15 | 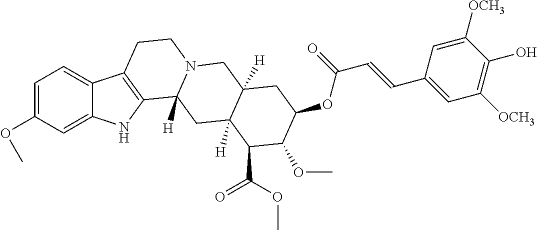 [SMILES: COC(=O)[C@H]1[C@H]2C[C@@H]3c4[nH]c5cc(OC)ccc5c4CCN3C[C@H]2C[C@@H](OC(=O)/C=C/c2cc(OC)c(O)c(OC)c2)[C@@H]1OC] | 47.67 | 54.53 |
| SA-I-029 | 12 | 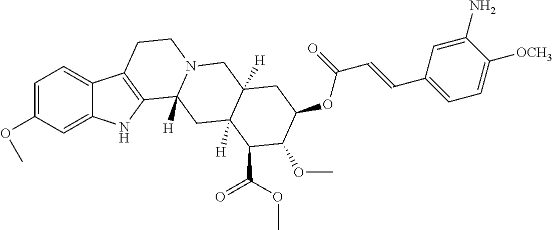 [SMILES: COC(=O)[C@H]1[C@H]2C[C@@H]3c4[nH]c5cc(OC)ccc5c4CCN3C[C@H]2C[C@@H](OC(=O)/C=C/c2ccc(OC)c(N)c2)[C@@H]1OC] | 77.25 | 113.4 |
| SA-I-039 | 13 | 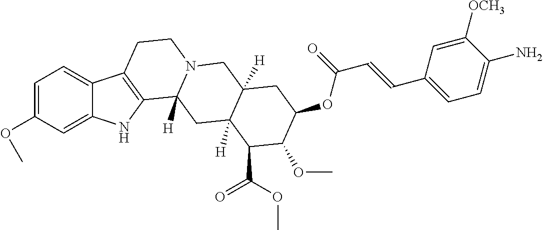 [SMILES: COC(=O)[C@H]1[C@H]2C[C@@H]3c4[nH]c5cc(OC)ccc5c4CCN3C[C@H]2C[C@@H](OC(=O)/C=C/c2ccc(N)c(OC)c2)[C@@H]1OC] | 85.73 | 68.38 |

NA: not applicable, since data did not reach below 50%.
“New/old” refers to known or unknown molecules.
IC50 values for individual compounds. Where appropriate, graphs that did not reach 50 & mortality were extrapolated. NA: no extrapolation possible

Additional examples of compounds of the present invention that can be prepared in like manner as described above, or variations thereof that will be apparent to persons skilled in the art, are given in Table 2:

TABLE 2

| Number | Compound |
|---|---|
| 81 | 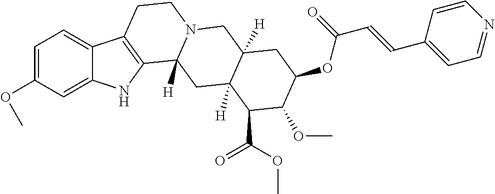 [SMILES: COC(=O)[C@H]1[C@H]2C[C@@H]3c4[nH]c5cc(OC)ccc5c4CCN3C[C@H]2C[C@@H](OC(=O)/C=C/c2ccncc2)[C@@H]1OC] |
| 88 | 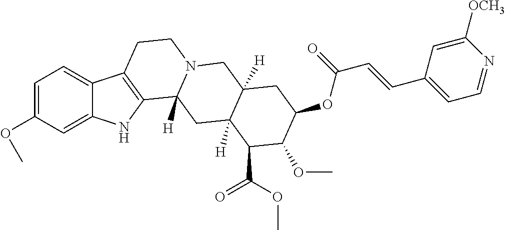 [SMILES: COC(=O)[C@H]1[C@H]2C[C@@H]3c4[nH]c5cc(OC)ccc5c4CCN3C[C@H]2C[C@@H](OC(=O)/C=C/c2ccnc(OC)c2)[C@@H]1OC] |
| 84 | 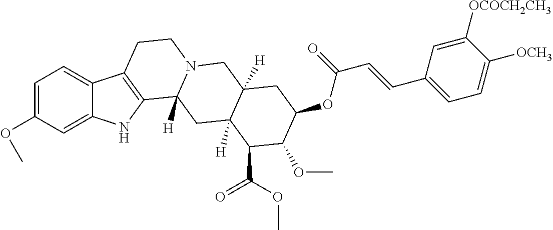 |
| 8 7 | 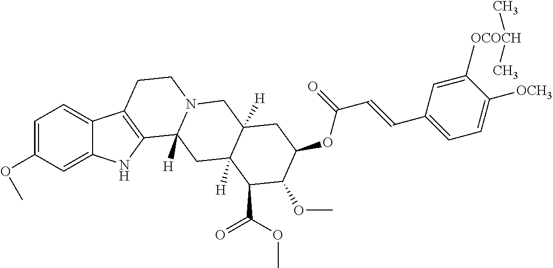 |
| 89 | 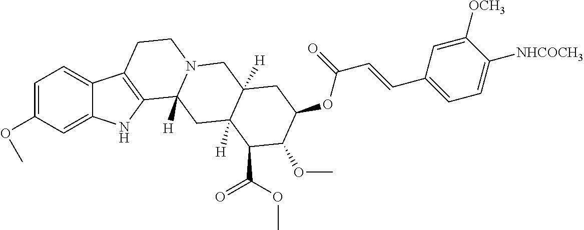 |
| 90 | 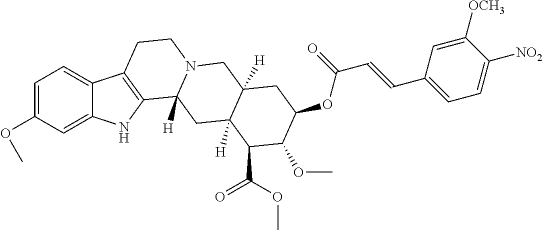 [SMILES: COC(=O)[C@H]1[C@H]2C[C@@H]3c4[nH]c5cc(OC)ccc5c4CCN3C[C@H]2C[C@@H](OC(=O)/C=C/c2ccc([N+](=O)[O-])c(OC)c2)[C@@H]1OC] |

Treatment of methyl reserpate, formed by the basic methanolysis of reserpine, with acryloyl chloride yields acryloyl reserpate (1) in 35% yield (Scheme 1). Similar treatment of 1 with propionyl chloride yields the saturated derivative 8 (Scheme 1). Exposure of 1 to various commercial or synthetic substituted aryl iodides (9) to palladium catalyzed Heck coupling conditions gives the di-substituted rescinnamine derivatives (2-7) in 27-68% yield and coupling constant analysis shows the exclusive formation of the trans stereoisomer (Scheme 1).

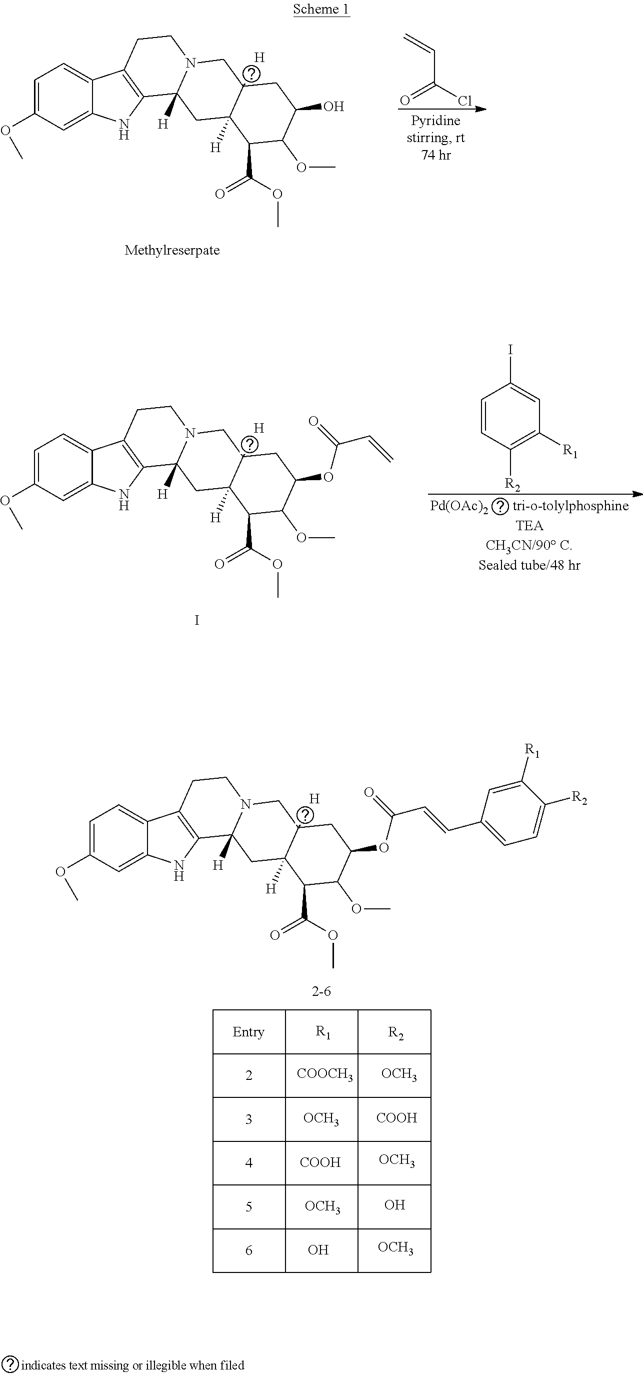

Condensation of the commercially available methyl esters with hydroxylamine produces the corresponding hydroxamic acids (10 and 11, Scheme 2) and Heck coupling of 1 to 10 and 11 in the absence of a phosphine ligand yields the aniline derivatives of rescinnamine (9 and 10, exclusive E stereochemistry) in 35 and 41% yield, respectively rather than the expected hydroxamic acids (Scheme 2). Such results suggest either a base or palladium-mediated Lossen rearrangement of these hydroxamic acid substrates, a process that holds some literature precedence. Similar Heck coupling of 1 to commercial tetra-substituted aryl iodides yields the tri-substituted rescinnamine derivatives 14 and 15 in 70 and 35% yield, respectively (Scheme 3). Extensive mass spectrometry and two-dimensional NMR experiments confirm the structure of 14, which lacks the expected p-methoxy group of the starting material (Scheme 3). A combination of proton and carbon nuclear magnetic (NMR) spectroscopy, mass spectrometry and elemental analysis confirms the identity of 1-15.

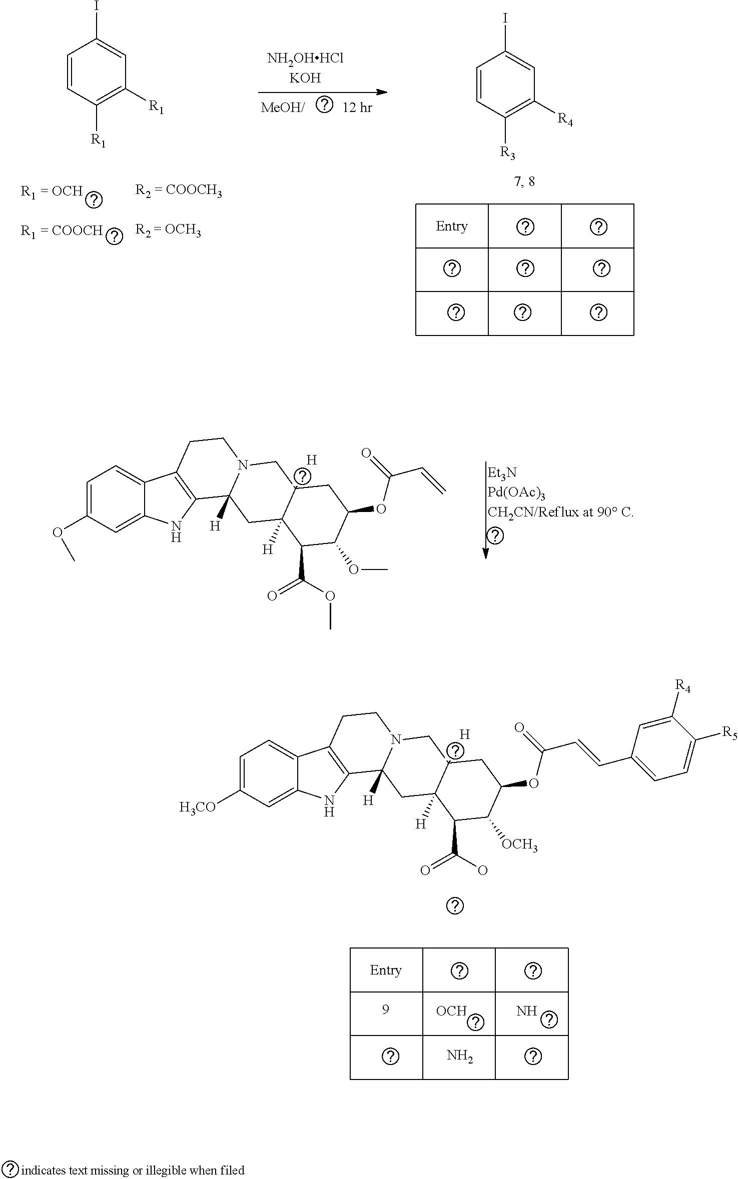

Effects of Rescinnamine Analogs on Cell Viability.

We next tested these new rescinnamine analogs in a well-defined cellular system with an endometrial cell line deficient (HEC59) and proficient (via chromosome transfer, HEC59+chr.2) for MSH2. These assays identified a few compounds that induced cell killing in the micromolar range (data not shown, compounds 03, 19, 20, 27, 39 to a lesser extent 29). However, little significant MSH2-dependence was observed for these compounds. The only compounds that demonstrated a small difference between MSH2-proficiency and deficiency were “12”, “16” and “39”. No significant difference was detected when determining the IC50 values. These results are surprising in-light of our previous work. Previous studies have suggested that in addition to MSH2-dependent cell-killing due to rescinnamine and derivatives, there is off-target cell-killing as well (Vasilyeva et al., 2010). These results suggest considerable off-target killing for these classes of compounds.

These results demonstrated that, although one compound even lacked an extra ring system, the nature of functional groups in key positions is critical, as altering these functional groups can lead to MSH2-independent cell-killing, or even to reduction in IC50 when MSH2 is present, such as with SA-I-013 and SA-I-029. These compound are of particular interest because the difference suggests some sort of interaction or effect due to MSH2, but one that does not lead to cell death.

Another surprising result is the drastic difference between isomers, SA-I-013 vs SA-I-015 especially, and also to a lesser event SA-I-029 vs SA-I-039. This difference, however, does suggest a preference for electron donating groups at R2 to avoid a reduction in IC50 when MSH2 is present. In SA-I-015 there is an electron donating group (OCH3) at R2, and an electron withdrawing group at R1. In SA-I-013, these groups are switched and there is an ˜8-fold change in sensitivity to MSH2. In SA-I-039 and SA-I-029, there are electron donating groups at both R1, and R2, but when the stronger electron donor is at R2, there is nearly a 2-fold change in sensitivity to MSH2.

### Cell Viability Assay

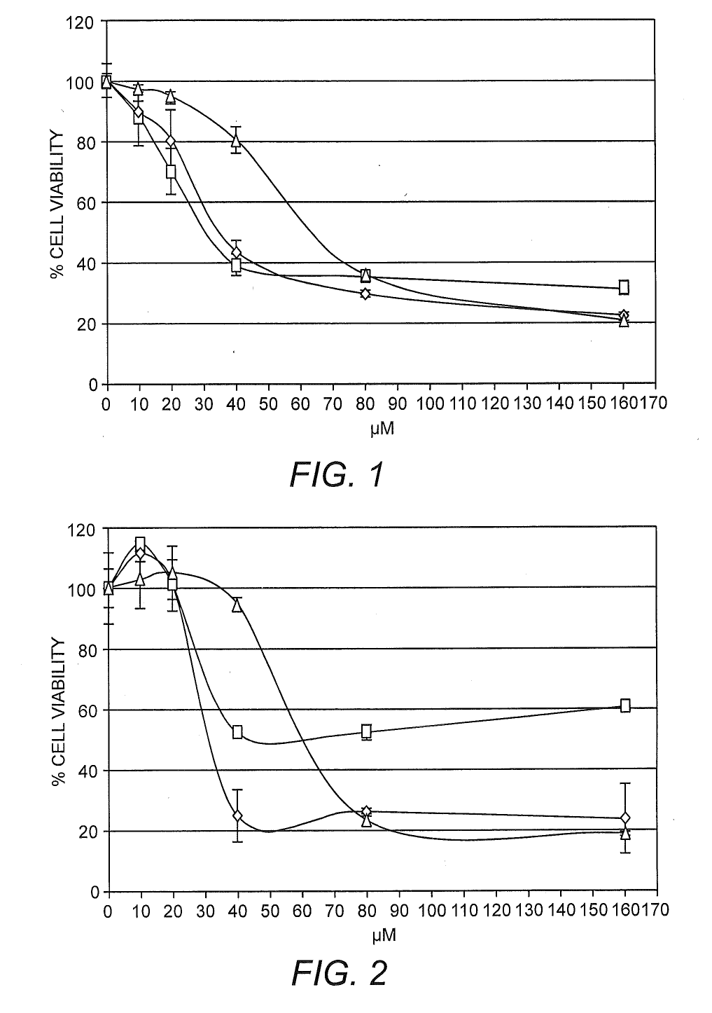

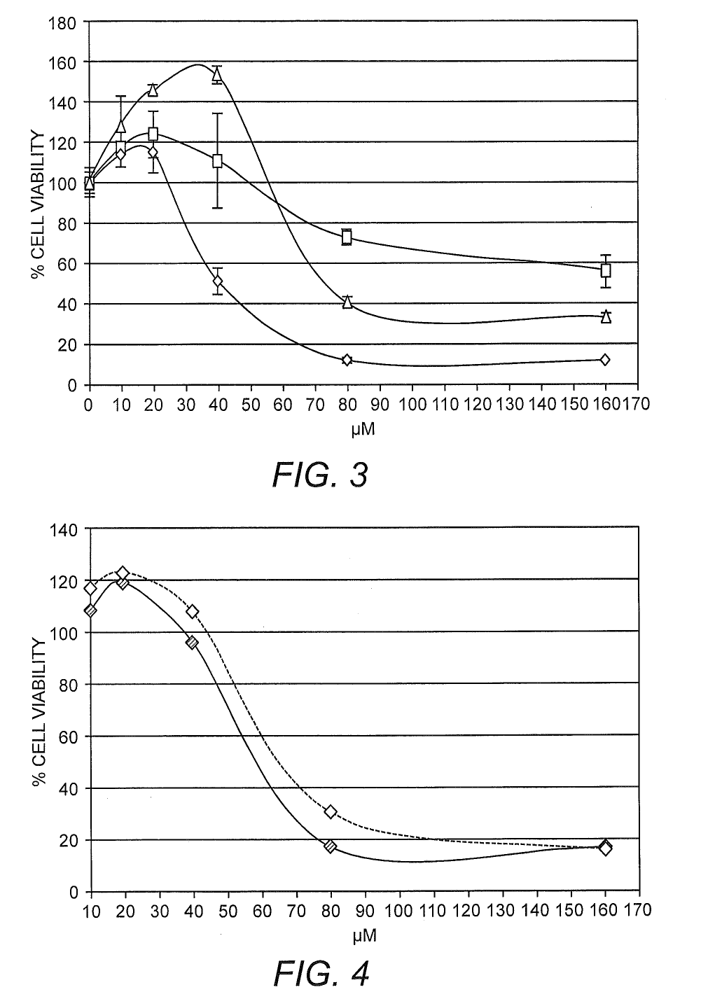

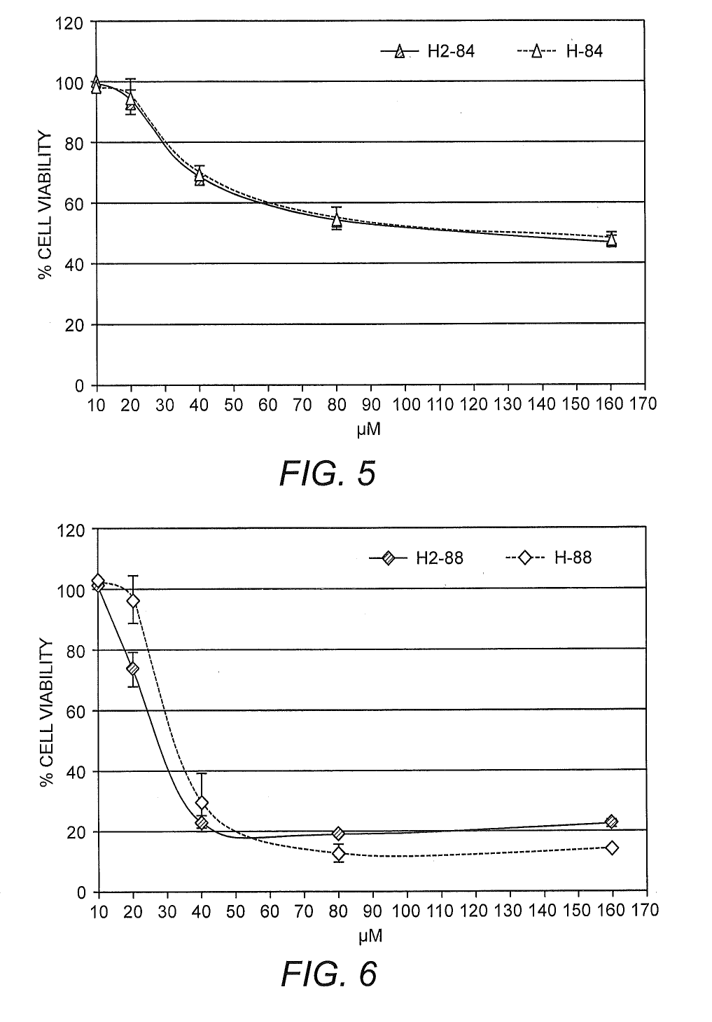

The activity of compounds 81, 84 and 88 in Table 2 was demonstrated in cell viability assays against various tumor cells. Briefly, cells were grown in standard growth media (DMEM-F12+10% FBS). Cells were plated in microtiter plates at an appropriate concentration in 100 microliter media and incubated overnight. Media was replaced with media containing drug and allowed to incubate for 24 hours at indicated concentrations. Untreated cells received fresh media with vehicle only. One solution reagent (CellTiter 96)(r) Aqueous One Solution) was added to existing media (20 microliters/well) and allowed to incubate 3-4 hrs. A plate reader was used to record the absorbance at 490 nm. Assays were performed at least in triplicates. Cell viability at each concentration was analyzed for IC₅₀ values using GraphPad Prism 4™. Data is given in FIGS. 1-6, where the graphs represent mean values and standard deviations.

### REFERENCES

Brambilla, G., and Martelli, A. (2006). Genotoxicity and carcinogenicity of antihypertensive agents. Mutat. Res. 612, 115-149.
Fife, R., MacLaurin, J. C., Wright, J. H. (1960). Rescinnamine in treatment of hypertension in hospital clinic and in general practice. Br. Med. J. 2, 1848-1850.
Morris, G. M., Huey, R., Lindstrom, W., Sanner, M. F., Belew, R. K., Goodsell, D. S, and Olson, A. J. (2009) Autodock4 and AutoDockTools4: automated docking with selective receptor flexiblity. J. Computational Chemistry, 16, 2785-91.
Morris, G. M., Goodsell, D. S., Halliday, R. S., Huey, R., Hart, W. E., Belew, R. K. and Olson, A. J. (1998), Automated Docking Using a Lamarckian Genetic Algorithm and Empirical Binding Free Energy Function J. Computational Chemistry, 19, 1639-1662.
Negureanu, L., and Salsbury Jr, F. R (2012). Insights into protein—DNA interactions, stability and allosteric communications: a computational study of mutSa-DNA recognition complexes. J Biomol Struct Dyn. 29, 757-76.
Salsbury Jr., F. R. (2010) Molecular dynamics simulations of protein dynamics and their relevance to drug discovery. Current opinion in pharmacology. 10, 738-44.
Salsbury Jr., F. R., Clodfelter, J. E., Gentry, M. B., Hollis, T., and Scarpinato, K. D. (2006). The molecular mechanism of DNA damage recognition by MutS homologs and its consequences for cell death response. Nucl. Acids Res. 34, 2173-2185.
Topping, R. P., Wilkinson, J. C., and Scarpinato, K. D. (2009). Mismatch repair protein deficiency compromises cisplatin-induced apoptotic signaling. J. Biol. Chem. 284, 14029-14039.
Umar, A., Koi, M., Risinger, J. I., Glaab, W. E., Tindall, K. R., Kolodner, R. D., Boland, C. R., Barrett, J. C., and Kunkel, T. A. (1997). Correction of hypermutability, N-Methyl-N′-nitro-N-nitrosoguanidine resistance, and defective DNA mismatch repair by introducing chromosome 2 into human tumor cells with mutations in MSH2 and MSH6. Cancer Res. 57, 3949-3955.
Vasilyeva, A., Clodfelter, J. E., Rector, B., Hollis, T., Scarpinato, K. D., and Salsbury Jr., F. R. (2009). Small molecule induction of MSH2-dependent cell death suggests a vital role of mismatch repair proteins in cell death. DNA Repair 8, 103-113.
Vasilyeva, A., Clodfelter, J. E., Gorczynski, M. J., Gerardi, A. R., King, S. B., Salsbury, F., and Scarpinato, K. D. (2010). Parameters of reserpine analogs that induce MSH2/MSH6-dependent cytotoxic response. J. Nucl. Acids 2010, 1-13.

The foregoing is illustrative of the present invention, and is not to be construed as limiting thereof. The invention is defined by the following claims, with equivalents of the claims to be included therein.

What is claimed is:

1. A compound of Formula I: 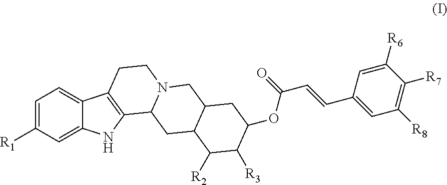
wherein:
R₁ is H, loweralkyl, or loweralkoxy;
R₂ is —C(O)OR″ or —NHC(O)OR″, where R″ is H or loweralkyl;
R₃ is hydroxyl or loweralkoxy;
R₆, R₇, and R₈ are each independently selected from the group consisting of H, OH, OCH₃, COON, —OC(O)R₉, amino, alkylamino, arylalkylamino, disubstituted amino, acylamino, amide, sulfoxyl, sulfonyl, sulfonate, sulfonic acid, and sulfonamide,
at least one of R₆, R₇ and R₈ is selected from the group consisting of nitro, —OC(O)R₉, amino, alkylamino, arylalkylamino, disubstituted amino, acylamino, amide, sulfoxyl, sulfonyl, sulfonate, sulfonic acid, and sulfonamide;
R₉ is loweralkyl,
or a pharmaceutically acceptable salt thereof.

2. A compound of claim 1 a structure selected from the group consisting of: 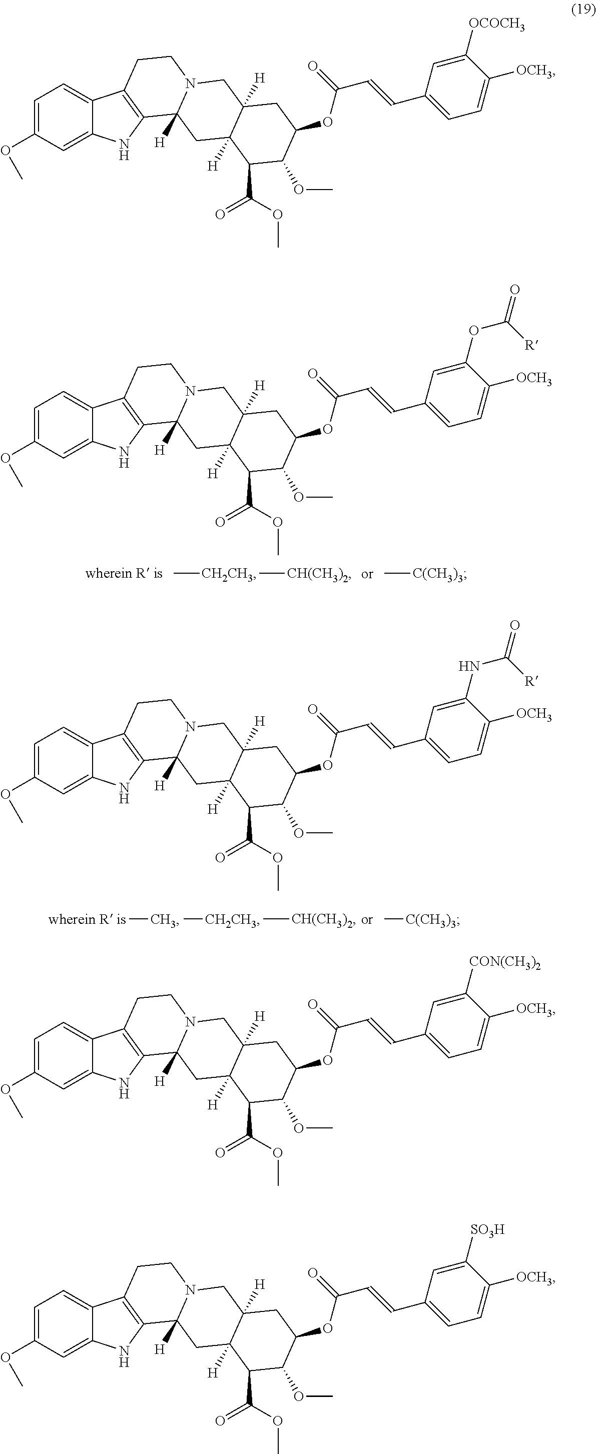 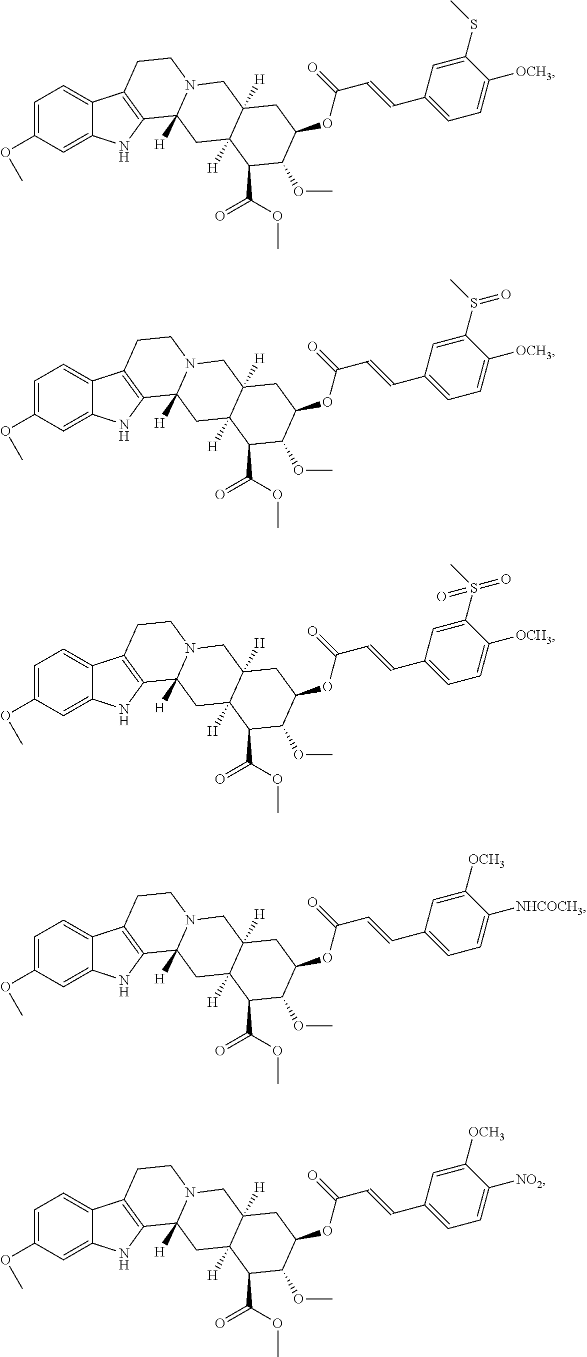
and pharmaceutically acceptable salts thereof.

3. A compound of Formula II: 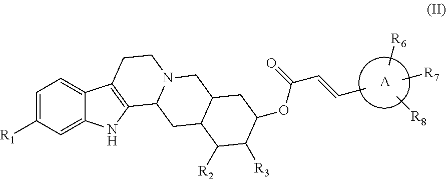
wherein:
ring A is an aromatic heterocyclic group;
R₁ is H, loweralkyl, or loweralkoxy;
R₂ is —C(O)OR″ or —NHC(O)OR″, where R″ is H or loweralkyl;
R₃ is hydroxyl or loweralkoxy;
R₆, R₇, and R₈ are each independently selected from the group consisting of H, OH, OCH₃, COON, —OC(O)R₉, amino, alkylamino, arylalkylamino, disubstituted amino, acylamino, amide, sulfoxyl, sulfonyl, sulfonate, sulfonic acid, and sulfonamide,
R₉ is loweralkyl,
or a pharmaceutically acceptable salt thereof.

4. The compound of claim 3, wherein ring A is selected from the group consisting of azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, and trithiane.

5. A compound of claim 3, wherein at least one of R₆, R₇ and R₈ is selected from the group consisting of —OC(O)R₉, amino, alkylamino, arylalkylamino, disubstituted amino, acylamino, amide, sulfoxyl, sulfonyl, sulfonate, sulfonic acid, and sulfonamide (preferably —OC(O)R₉, amino, alkylamino, arylalkylamino, disubstituted amino, acylamino, or amide).

6. A compound of claim 3 having the structure of selected from the group consisting of:

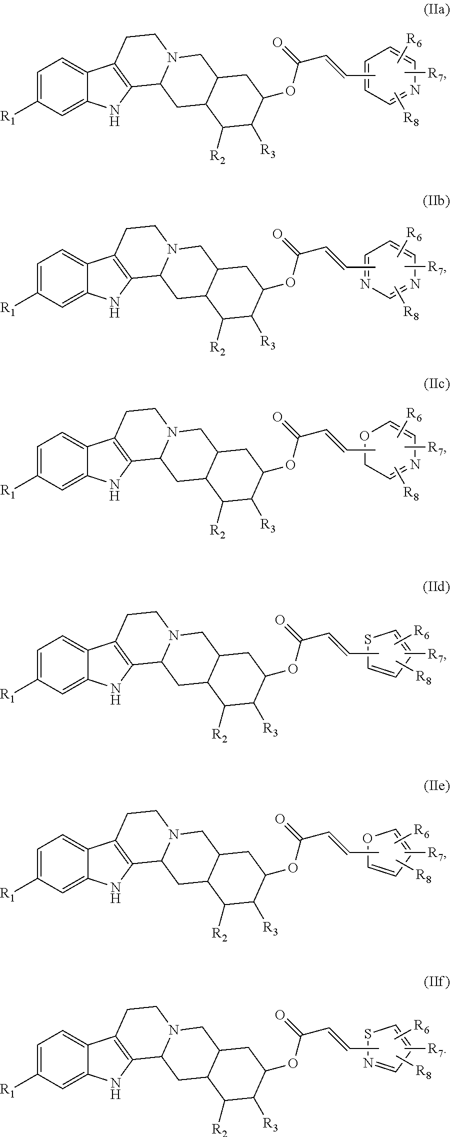

7. A compound of claim 3 selected from the group consisting of: 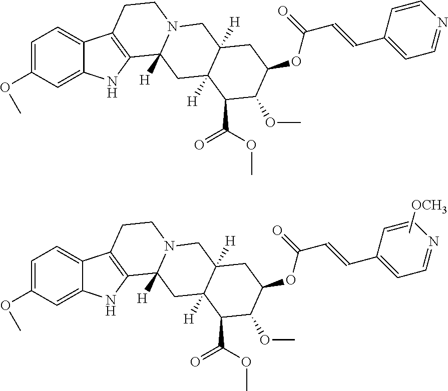
and pharmaceutically acceptable salts thereof.

8. A compound of claim 1, wherein:
R₁ is methoxy; and
R₂ is —C(O)OR″ where R″ is H or methyl.

9. A compound of the formula: 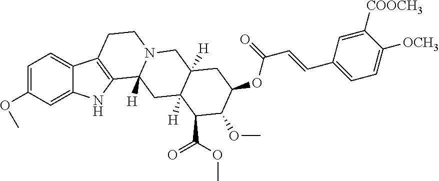 [SMILES: COC(=O)c1cc(/C=C/C(=O)O[C@@H]2C[C@@H]3CN4CCc5c([nH]c6cc(OC)ccc56)[C@H]4C[C@@H]3[C@H](C(=O)OC)[C@H]2OC)ccc1OC]
or a pharmaceutically acceptable salt thereof.

10. A method of treating cancer in a subject in need thereof, comprising administering said subject an active agent in an amount effective to treat said cancer, wherein said active agent is a compound of claim 1.

11. The method of claim 10, wherein said cancer is selected from the group consisting of lung cancer, colon cancer, esophageal cancer, ovarian cancer, skin cancer, and gastric cancer.

12. The method of claim 10, wherein said cancer is selected from the group consisting of lung cancer and colon cancer.

13. The method of claim 10, wherein said cancer comprises a p53-deficient tumor.

14. The method of claim 10, wherein said active agent is administered in combination with radiation therapy.

15. The method of claim 10, wherein said active agent is administered in combination with ablative or partially ablative surgery.

16. The method of claim 10, wherein said active agent is administered in combination with one or more additional chemotherapeutic agents.

17. The method of claim 16, wherein said additional chemotherapeutic agent is selected from the group consisting of androgens, asparaginase, azathioprine, 5-azacitidine, BCG, bleomycin, busulfan, carbetimer, carboplatin chlorambucil, cisplatin, corticosteroids, cyclophosphamide, cytarabine, dacarbazine, dactinomycin, daunomycin, doxorubicin, epirubicin, estrogens, etoposide, fadrazole, 5-fluorouracil, gemcitabine, hydroxyurea, ifosfamide, interferon alpha, interferon beta, interferon gamma, an interleukin, isotretinoin, lomustine, melphalan, 6-mercaptopurine, methotrexate, mitomycin-c, mitotane, mitoxantrone, paclitaxel, pentostatin, procabazine, progestins, rituximab, streptozocin, tamoxifen, taxotere, teniposide, thioguanine, thiotepa, topotecan, toremifene, tretinoin, uracil mustard, vinblastine, vincristine and vinorelbine.

18. A composition comprising a compound of claim 1 in a pharmaceutically acceptable carrier.

19. The composition of claim 18, further comprising at least one additional chemotherapeutic agent.

20. The composition of claim 19, wherein said additional chemotherapeutic agent is selected from the group consisting of androgens, asparaginase, azathioprine, 5-azacitidine, BCG, bleomycin, busulfan, carbetimer, carboplatin chlorambucil, cisplatin, corticosteroids, cyclophosphamide, cytarabine, dacarbazine, dactinomycin, daunomycin, doxorubicin, epirubicin, estrogens, etoposide, fadrazole, 5-fluorouracil, gemcitabine, hydroxyurea, ifosfamide, interferon alpha, interferon beta, interferon gamma, an interleukin, isotretinoin, lomustine, melphalan, 6-mercaptopurine, methotrexate, mitomycin-c, mitotane, mitoxantrone, paclitaxel, pentostatin, procabazine, progestins, rituximab, streptozocin, tamoxifen, taxotere, teniposide, thioguanine, thiotepa, topotecan, toremifene, tretinoin, uracil mustard, vinblastine, vincristine and vinorelbine.
